(19) United States
(12) Patent Application Publication
(10) Pub. No.: US 2009/0222097 A1
(43) Pub. Date: Sep. 3, 2009

(54) Nucleus implant and method of installing same

(75) Inventors: Mingyan Liu, Bourg la Reine (FR); Jean-Charles LeHuec, Pessac (FR); Kenneth J. Burkus, Columbus, GA (US); Loic Josse, Denens (FR)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/038,987

(22) Filed: Feb. 28, 2008

(51) Int. Cl. A61F 2/44

(52) U.S. Cl. 623/17.16; 623/17.11; 623/17.12

(57) ABSTRACT

A nucleus implant is disclosed. The nucleus implant can be configured to be installed within an intervertebral disc between an inferior vertebra and a superior vertebra. The nucleus implant can include an expandable core and an expandable chamber that can be disposed at least partially around the expandable core. The expandable chamber can be expanded from a deflated position to an inflated position. Further, a hardness of the expandable core when inflated can greater than or equal to a hardness of the expandable chamber when inflated.

### FIELD OF THE DISCLOSURE

The present disclosure relates generally to orthopedics and spinal surgery. More specifically, the present disclosure relates to nucleus implants.

### BACKGROUND

In human anatomy, the spine is a generally flexible column that can take tensile and compressive loads. The spine also allows bending motion and provides a place of attachment for ribs, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of “wear and tear”.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively. Additionally, it is known to surgically remove nucleus pulposus material from within an intervertebral disc and replace the nucleus pulposus material with an artificial nucleus.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a cross section view of an intervertebral disc;

FIG. 5 is a plan view of a first embodiment of a nucleus implant;

FIG. 6 is another plan view of the first embodiment of the nucleus implant;

FIG. 7 is a cross-section view of the first embodiment of the nucleus implant taken along line 7-7 in FIG. 6;

FIG. 8 is a plan view of a second embodiment of a nucleus implant;

FIG. 9 is another plan view of the second embodiment of the nucleus implant;

FIG. 10 is a cross-section view of the second embodiment of the nucleus implant taken along line 10-10 in FIG. 9;

FIG. 11 is a cross-section of a first embodiment of a set of nucleus implant injection tubes;

FIG. 12 is a cross-section of a second embodiment of a set of nucleus implant injection tubes;

FIG. 13 is a cross-section of a third embodiment of a set of nucleus implant injection tubes;

FIG. 14 is a flow chart of a first method of installing a nucleus implant;

FIG. 15 is a plan view of a third embodiment of a nucleus implant;

FIG. 16 is another plan view of the third embodiment of the nucleus implant;

FIG. 17 is a cross-section view of the third embodiment of the nucleus implant taken along line 17-17 in FIG. 16;

FIG. 18 is a cross-section of a fourth embodiment of a set of nucleus implant injection tubes;

FIG. 19 is a cross-section of a fifth embodiment of a set of nucleus implant injection tubes;

FIG. 20 is a cross-section of a sixth embodiment of a set of nucleus implant injection tubes;

FIG. 21 is a cross-section of a seventh embodiment of a set of nucleus implant injection tubes;

FIG. 22 is a cross-section of an eighth embodiment of a set of nucleus implant injection tubes;

FIG. 23 is a flow chart of a second method of installing a nucleus implant;

FIG. 24 is a plan view of a fourth embodiment of a nucleus implant;

FIG. 25 is another plan view of the fourth embodiment of the nucleus implant;

FIG. 26 is a cross-section view of the fourth embodiment of the nucleus implant taken along line 26-26 in FIG. 25; and

FIG. 27 is a flow chart of a third method of installing a nucleus implant.

### DETAILED DESCRIPTION OF THE DRAWINGS

A nucleus implant is disclosed. The nucleus implant can be configured to be installed within an intervertebral disc between an inferior vertebra and a superior vertebra. The nucleus implant can include an expandable core and an expandable chamber that can be disposed at least partially around the expandable core. The expandable chamber can be expanded from a deflated position to an inflated position. Further, a hardness of the expandable core, when inflated, can be greater than or equal to a hardness of the expandable chamber when inflated.

It will be noted that the chamber that is at least partially peripheral enables, when it is inflated, accurate positioning of the core. This implant provides a great mobility from one vertebra to another vertebra (rotation and/or flexion). Since the core is harder than the chamber it acts as a pivot, thereby making these movements easier.

In another embodiment, a nucleus implant is disclosed. The nucleus implant can be configured to be installed within an intervertebral disc between an inferior vertebra and a superior vertebra. The nucleus implant can include an expandable core and a toroid shaped expandable chamber that can be disposed at least partially around the expandable core. Moreover, a hardness of the expandable core, when inflated, can be greater or equal to than a hardness of the toroid shaped expandable chamber when inflated.

In yet another embodiment, a nucleus implant is disclosed. The nucleus implant can be configured to be installed within an intervertebral disc between an inferior vertebra and a superior vertebra. The nucleus implant can include an expandable core that can include an outer surface. Also, the nucleus implant can include a first toroid shaped expandable chamber that can be disposed at least partially around the expandable core and a second toroid shaped expandable chamber that can be disposed at least partially around the first toroid shaped expandable chamber. A hardness of the expandable core can be greater than or equal to a hardness of the first expandable chamber and the hardness of the first expandable chamber can be greater than or equal to a hardness of the second expandable chamber when the expandable core, the first expandable chamber, and the second expandable chamber are inflated.

In still another embodiment, a nucleus implant is disclosed. The nucleus implant can be configured to be installed within an intervertebral disc between an inferior vertebra and a superior vertebra. The nucleus implant can include an expandable core and a bowl shaped expandable chamber that can be disposed at least partially around the expandable core. A hardness of the expandable core when inflated can be greater than or equal to a hardness of the bowl shaped expandable chamber when inflated.

In yet still another embodiment, a nucleus implant is disclosed. The nucleus implant can be configured to be installed within an intervertebral disc between an inferior vertebra and a superior vertebra. The nucleus implant can include an expandable core that can include an outer surface and a U shaped expandable chamber that can be disposed at least partially around the expandable core. A hardness of the expandable core when inflated can be greater than or equal to a hardness of the U shaped expandable chamber when inflated.

In another embodiment, a method of installing a nucleus implant within an intervertebral disc between an inferior vertebra and a superior vertebra of a patient is disclosed. The method can include implanting the nucleus implant within the intervertebral disc. Further, the nucleus implant can include an expandable core and an expandable chamber at least partially around the expandable core. The method can also include inflating the expandable core and inflating the expandable chamber around the expandable core. A hardness of the expandable core when inflated can be greater than or equal to a hardness of the expandable chamber when inflated.

In yet another embodiment, a method of installing a nucleus implant within an intervertebral disc between an inferior vertebra and a superior vertebra of a patient is disclosed. The method can include implanting the nucleus implant within the intervertebral disc. The nucleus implant can include an expandable core, a first expandable chamber at least partially around the expandable core, and a second expandable chamber at least partially around the first expandable chamber. Moreover, the method can inflating the expandable core, inflating the first expandable chamber around the expandable core, inflating the second expandable chamber around the first expandable chamber. A hardness of the expandable core when inflated can be greater than or equal to a hardness of the first expandable chamber when inflated. Also, the hardness of the first expandable chamber when inflated can be greater than or equal to a hardness of the second expandable chamber when inflated.

In still yet another embodiment, a method of installing a nucleus implant within an intervertebral disc between an inferior vertebra and a superior vertebra of a patient is disclosed. The method can include implanting the nucleus implant within the intervertebral disc. The nucleus implant can include an expandable core and an expandable chamber at least partially around the expandable core. Additionally, the method can include inflating the expandable chamber and inflating the expandable core within the expandable chamber. A hardness of the expandable core when inflated can be greater than or equal to a hardness of the expandable chamber when inflated.

### Description of Relevant Anatomy

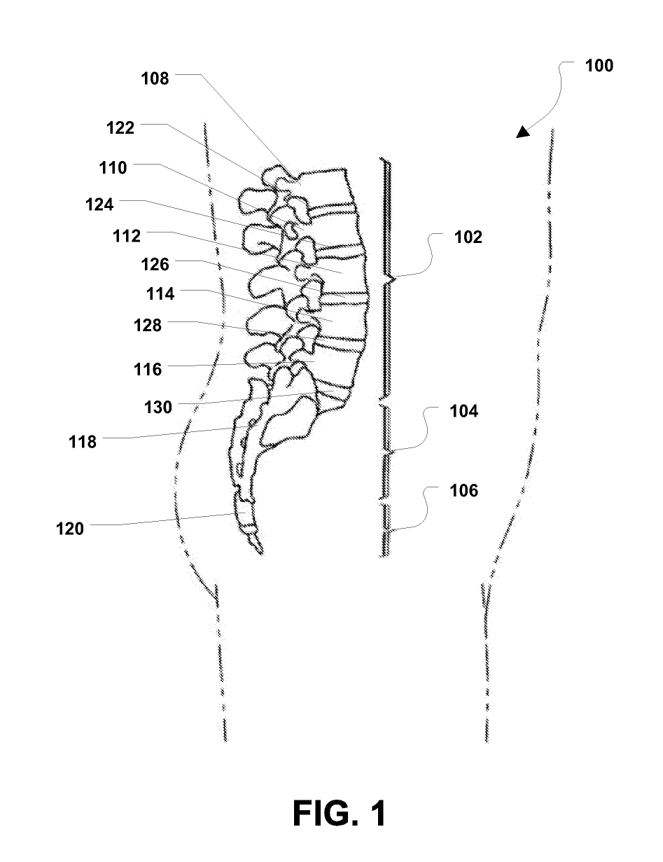

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

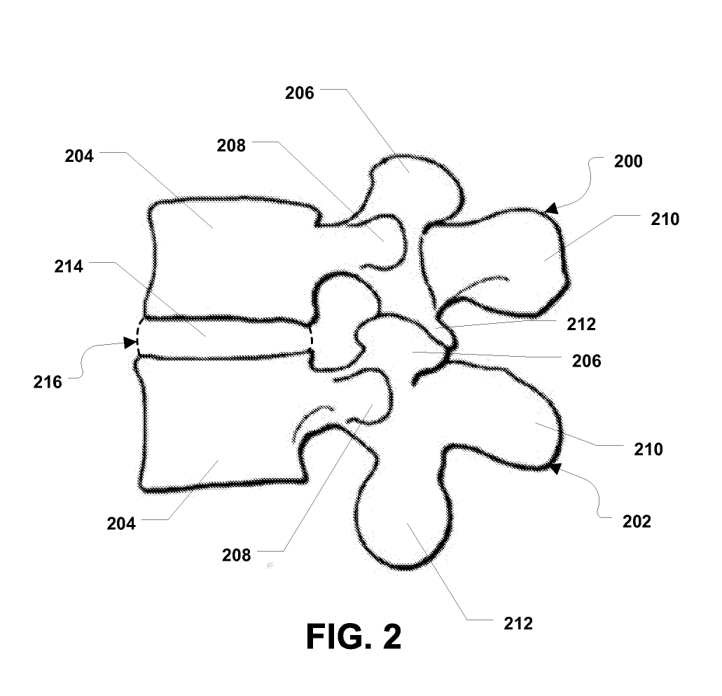

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines).

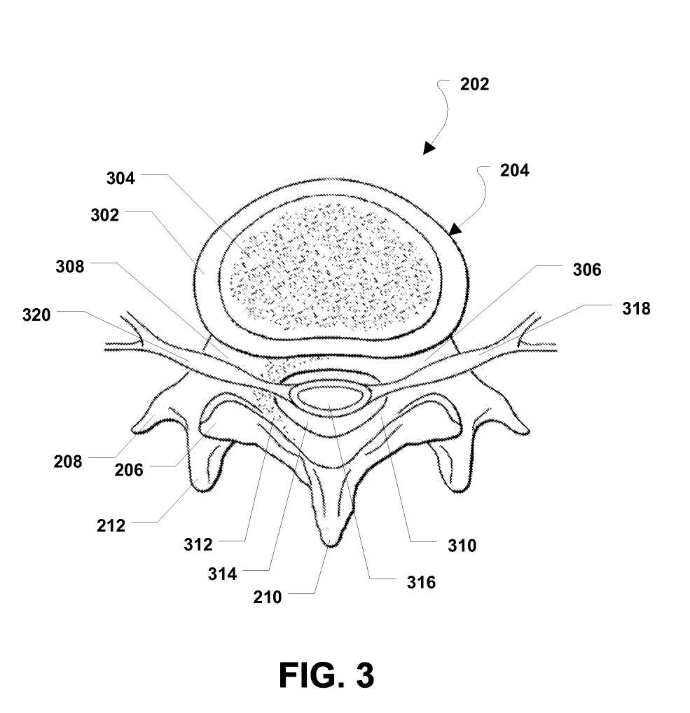

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer and weaker than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

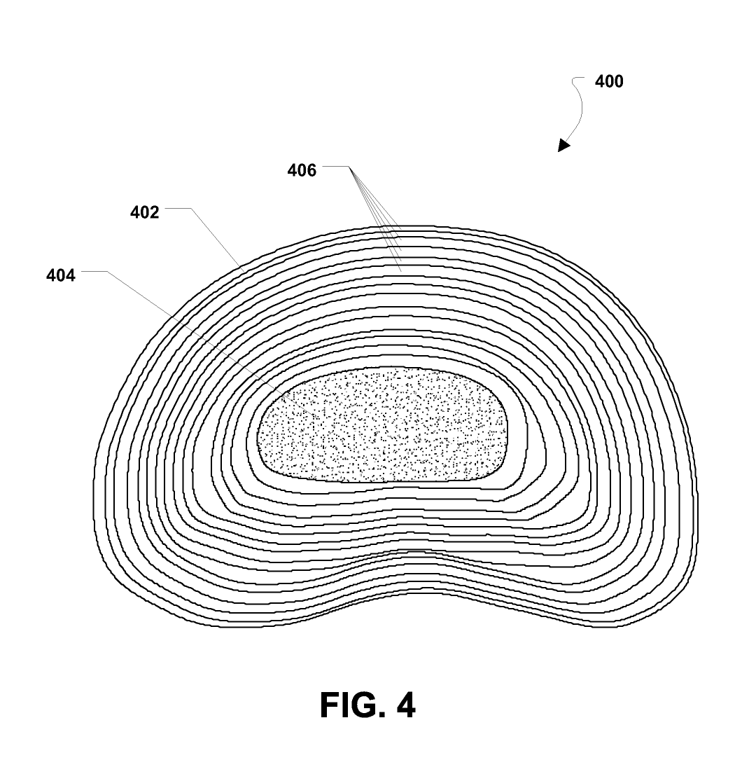

Referring now to FIG. 4, an intervertebral disc is shown and is generally designated 400. The intervertebral disc 400 is made up of two components: the annulus fibrosus 402 and the nucleus pulposus 404. The annulus fibrosus 402 is the outer portion of the intervertebral disc 400, and the annulus fibrosus 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosus 402 is a structure that is exceptionally strong, yet extremely flexible.

The nucleus pulposus 404 is the inner gel material that is surrounded by the annulus fibrosus 402. It makes up about forty percent (40%) of the intervertebral disc 400. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) at birth and decreases to about seventy percent (70%) by the fifth decade.

Injury or aging of the annulus fibrosus 402 may allow the nucleus pulposus 404 to be squeezed through the annulus fibers either partially, causing the disc to bulge, or completely, allowing the disc material to escape the intervertebral disc 400. The bulging disc or nucleus material may compress the nerves or spinal cord, causing pain. Accordingly, the nucleus pulposus 404 can be removed and replaced with an artificial nucleus.

### DESCRIPTION OF A FIRST EMBODIMENT OF A NUCLEUS IMPLANT

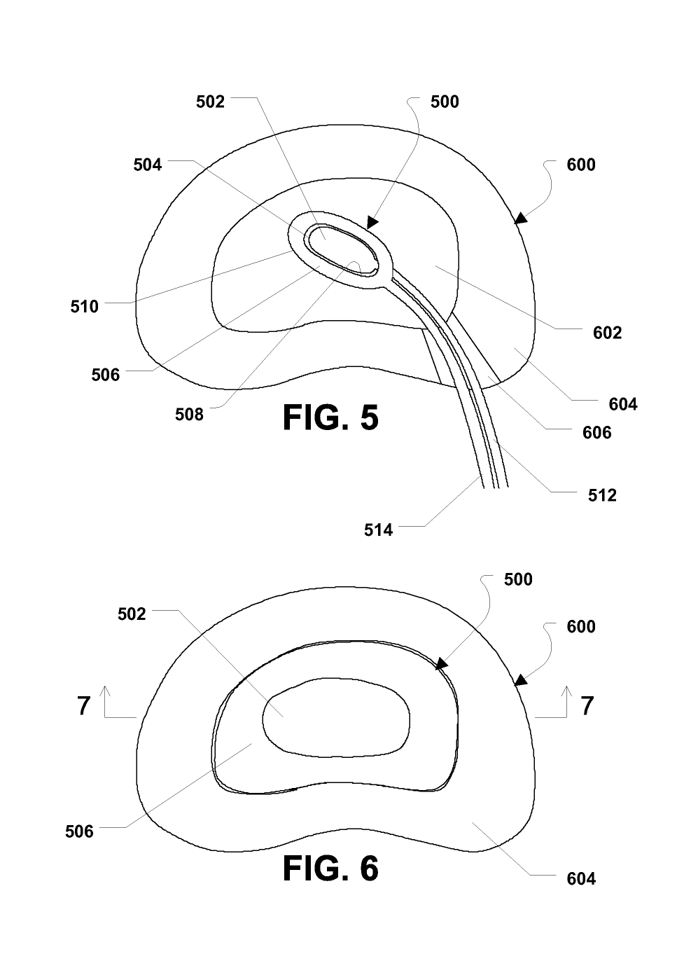

Referring to FIG. 5 through FIG. 7, an embodiment of a nucleus implant is shown and is designated 500. As shown, the nucleus implant 500 includes an expandable core 502 that defines an outer surface 504. In a particular embodiment, when inflated, the expandable core 502 can have a cross-section that is generally elliptical. Alternatively, the expandable core 502 can have a cross-section that is: generally circular, generally rectangular, generally square, generally triangular, generally trapezoidal, generally rhombic, generally quadrilateral, any generally polygonal shape, or any combination thereof.

As illustrated in FIG. 5 and FIG. 6, an expandable chamber 506 can be disposed around the expandable core 502. In a particular embodiment, as shown, the expandable chamber 506 can have a generally toroidal shape. The shape of the chamber enables, when expanded or inflated, automatic positioning of the core. Further, when expanded, or inflated, the expandable chamber 506 can have a cross-section that is generally shaped like a kidney bean. Alternatively, the expandable chamber 506 can have a cross-section that is: generally elliptical, generally circular, generally rectangular, generally square, generally triangular, generally trapezoidal, generally rhombic, generally quadrilateral, any generally polygonal shape, or any combination thereof.

The expandable chamber 506 can define an inner surface 508 and an outer surface 510. In a particular embodiment, the inner surface 508 of the expandable chamber 506 can be attached to the outer surface 504 of the expandable core 502, for example, by gluing. As such, proper placement of the expandable chamber 506 can be based on the placement of the expandable core 502. Alternatively, the expandable chamber 506 can be separate from the expandable core 502 and the expandable chamber 506 may engage the expandable core 502 after the expandable chamber 506 is properly inflated. Alternatively, the core and the chamber may be made of one and the same element, for example, for the sake of convenience.

As depicted in FIG. 5, the nucleus implant can include a first injection tube 512 that extends from the outer surface 504 of the expandable core 502. Further, the nucleus implant 500 can include a second injection tube 514 that extends from the outer surface 510 of the expandable chamber 506. In a particular embodiment, each of the expandable core 502 and the expandable chamber 506 of the nucleus implant 500 is expandable from a respective deflated position, shown in FIG. 5, to one selected position among a plurality of inflated positions, shown in FIG. 6, up to a maximum inflated position. Further, after the expandable core 502 and the expandable chamber 506 are inflated, or otherwise expanded, the injection tubes 512, 514 can be removed, as depicted in FIG. 6.

In a particular embodiment, the nucleus implant 500 can include a first self-sealing valve (not shown) within the outer surface 504 of the expandable core 502, e.g., adjacent to the first injection tube 512. Moreover, the nucleus implant 500 can include a second self-sealing valve (not shown) within the outer surface 510 of the expandable chamber 506, e.g., adjacent to the second injection tube 514. The self-sealing valves can prevent the expandable core 502 and the expandable chamber 506 from leaking material after the expandable core 502 and the expandable chamber 506 are inflated and the injection tubes 512, 514 are removed.

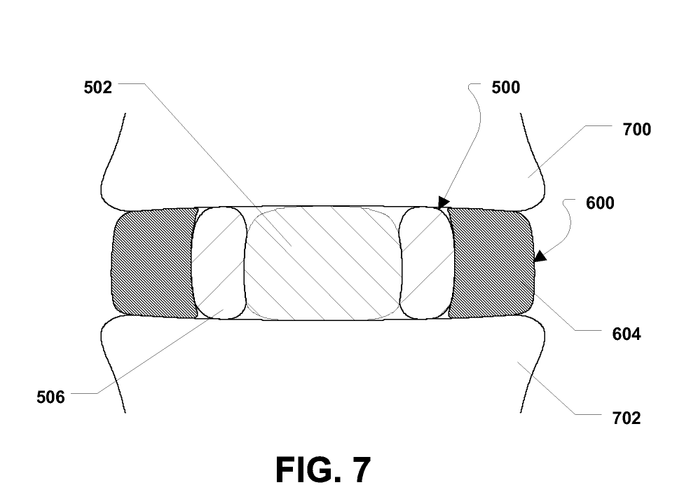

FIG. 7 indicates that the nucleus implant 500 can be implanted within an intervertebral disc 600 between a superior vertebra 700 and an inferior vertebra 702. More specifically, the nucleus implant 500 can be implanted within an intervertebral disc space 602 established within the annulus fibrosus 604 of the intervertebral disc 600. The intervertebral disc space 602 can be established by removing the nucleus pulposus (not shown) from within the annulus fibrosus 604.

In a particular embodiment, the expandable core 502 and the expandable chamber 506 can be inflated so the inner surface 508 of the expandable chamber 506 engages the outer surface of the expandable core 502 and the outer surface 510 of the expandable chamber 506 engages the annulus fibrosis 604. The nucleus implant 500 can provide shock-absorbing characteristics substantially similar to the shock absorbing characteristics provided by the nucleus pulposus. Further, in a particular embodiment, the hardness of the expandable core 502 of the nucleus implant 500 is greater than or equal to the hardness of the material used to inflate the expandable chamber 506, i.e., after the materials used to inflate the expandable core 502 and the expandable chamber 506 are cured. Alternatively, the viscosity of the material used to inflate the expandable core 502 is greater than or equal to the viscosity of the material used to inflate the expandable chamber 506. As one example, the core has a hardness of 55 Shore D and the expandable chamber has a hardness of 40 Shore D.

Additionally, in a particular embodiment, the height of the expandable core 502 is greater than or equal to the height of the expandable chamber 506 when each is properly expanded within the intervertebral disc 600. As shown in FIG. 7, the expandable core 502 and the expandable chamber 506 of the nucleus implant 500 can be configured to provide proper support and spacing between the superior vertebra 700 and the inferior vertebra 702.

In a particular embodiment, the expandable core 502, the expandable chamber 506, or both the expandable core 502 and the expandable chamber 506 of the nucleus implant 500 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the expandable core 502, the expandable chamber 506, or both the expandable core 502 and the expandable chamber 506 of the nucleus implant 500 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 500 can be installed using a posterior surgical approach, as shown. Further, the nucleus implant 500 can be installed through a posterior incision 606 made within the annulus fibrosus 604 of the intervertebral disc 600. Alternatively, the nucleus implant 500 can be installed using an anterior surgical approach, a lateral surgical approach, or any other surgical approach well known in the art.

### DESCRIPTION OF A SECOND EMBODIMENT OF A NUCLEUS IMPLANT

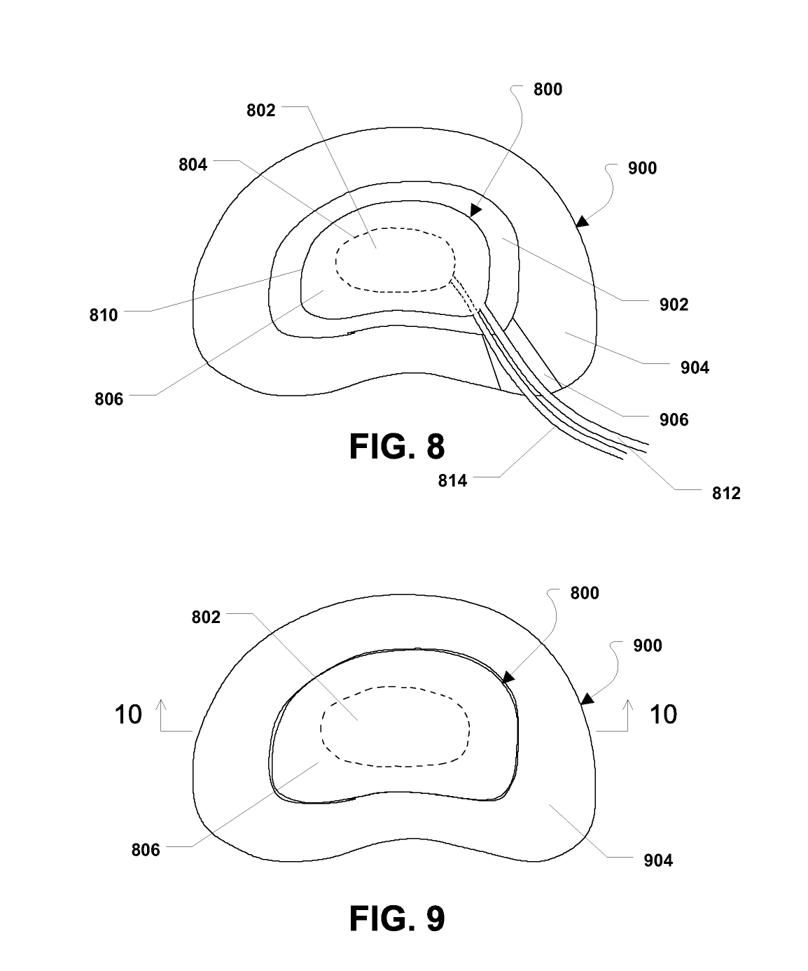

Referring to FIG. 8 through FIG. 10, a second embodiment of a nucleus implant is shown and is designated 800. As shown, the nucleus implant 800 includes an expandable core 802 that defines an outer surface 804. In a particular embodiment, when inflated, or otherwise expanded, the expandable core 802 can have a cross-section that is generally elliptical. Alternatively, the expandable core 802 can have a cross-section that is: generally circular, generally rectangular, generally square, generally triangular, generally trapezoidal, generally rhombic, generally quadrilateral, any generally polygonal shape, or any combination thereof.

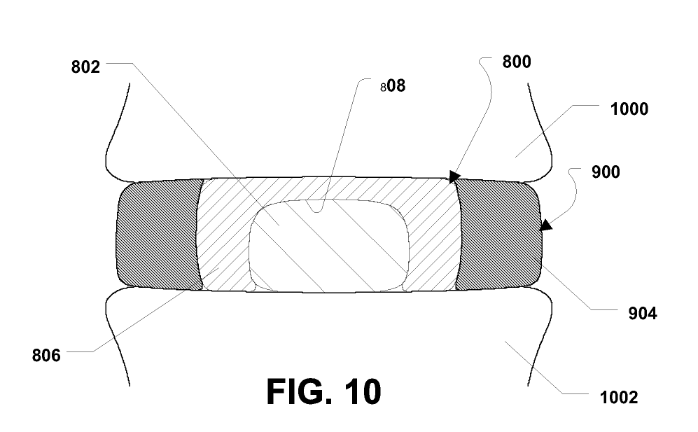

As illustrated in FIG. 8 through FIG. 10, an expandable chamber 806 can be disposed around the expandable core 802. In a particular embodiment, when expanded, or otherwise inflated, the expandable chamber 806 can have a generally inverted-bowl shape and the expandable chamber 806 can be draped, or otherwise placed, over the expandable core 802 as shown in FIG. 10.

The thus shaped chamber that is arranged around the core may enable accurate positioning of the core. The accuracy of the core positioning may be increased by inflating the chamber with a uniform pressure.

The expandable chamber 806 can define an inner surface 808 and an outer surface 810. In a particular embodiment, the inner surface 808 of the expandable chamber 806 can be attached to the outer surface 804 of the expandable core 802. As such, proper placement of the expandable chamber 806 can be based on the placement of the expandable core 802. Alternatively, the expandable chamber 806 can be separate from the expandable core 802 and the expandable chamber 806 may engage the expandable core 802 after the expandable chamber 806 and the expandable core 802 are properly inflated.

As depicted in FIG. 8, the nucleus implant 800 can include a first injection tube 812 that extends from the outer surface 804 of the expandable core 802. Further, the nucleus implant 800 can include a second injection tube 814 that extends from the outer surface 810 of the expandable chamber 806. In a particular embodiment, each of the expandable core 802 and the expandable chamber 806 of the nucleus implant 800 is expandable from a deflated position, shown in FIG. 8, to one selected position among a plurality of inflated positions, shown in FIG. 9, up to a maximum inflated position. Further, after the expandable core 802 and the expandable chamber 806 are inflated, or otherwise expanded, the injection tubes 812, 814 can be removed, as depicted in FIG. 9.

In a particular embodiment, the nucleus implant 800 can include a first self-sealing valve (not shown) within the outer surface 804 of the expandable core 802, e.g., adjacent to the first injection tube 812. Moreover, the nucleus implant 800 can include a second self-sealing valve (not shown) within the outer surface 810 of the expandable chamber 806, e.g., adjacent to the second injection tube 814. The self-sealing valves can prevent the expandable core 802 and the expandable chamber 806 from leaking material after the expandable core 802 and the expandable chamber 806 are inflated and the injection tubes 812, 814 are removed.

FIG. 10 indicates that the nucleus implant 800 can be implanted within an intervertebral disc 900 between a superior vertebra 1000 and an inferior vertebra 1002. More specifically, the nucleus implant 800 can be implanted within an intervertebral disc space 902 established within the annulus fibrosus 904 of the intervertebral disc 900. The intervertebral disc space 902 can be established by removing the nucleus pulposus (not shown) from within the annulus fibrosus 904.

In a particular embodiment, the expandable core 802 and the expandable chamber 806 can be inflated so the inner surface 808 of the expandable chamber 806 engages the outer surface of the expandable core 802 and the outer surface 810 of the expandable chamber 806 engages the annulus fibrosis 904. Further, portions of the outer surface 810 of the expandable chamber 806 can engage the superior vertebra 1000 and an inferior vertebra 1002. Moreover, when the expandable core 802 and the expandable chamber 806 are expanded, or otherwise inflated, a portion of the expandable chamber 806 is located between the expandable core 802 and the superior vertebra 1000.

The nucleus implant 800 can provide shock-absorbing characteristics substantially similar to the shock absorbing characteristics provided by the nucleus pulposus. Further, in a particular embodiment, the hardness of the expandable core 802 of the nucleus implant 800 is greater than or equal to the hardness of the material used to inflate the expandable chamber 806, i.e., after the materials used to inflate the expandable core 802 and the expandable chamber 806 are cured. Alternatively, the viscosity of the material used to inflate the expandable core 802 is greater than or equal to the material used to inflate the expandable chamber 806. As shown in FIG. 10, the expandable core 802 and the expandable chamber 806 of the nucleus implant 800 can be configured to provide proper support and spacing between the superior vertebra 1000 and the inferior vertebra 1002.

In a particular embodiment, the expandable core 802, the expandable chamber 806, or both the expandable core 802 and the expandable chamber 806 of the nucleus implant 800 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the expandable core 802, the expandable chamber 806, or both the expandable core 802 and the expandable chamber 806 of the nucleus implant 800 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 800 can be installed using a posterior surgical approach, as shown. Further, the nucleus implant 800 can be installed through a posterior incision 906 made within the annulus fibrosus 904 of the intervertebral disc 900. When the chamber has been inflated, the core is not able to get out through the incision 906 after its installation. This is because the bottom of the chamber having a generally inverted-bowl shape is placed between the incision and the core. Alternatively, the nucleus implant 800 can be installed using an anterior surgical approach, a lateral surgical approach, or any other surgical approach well known in the art.

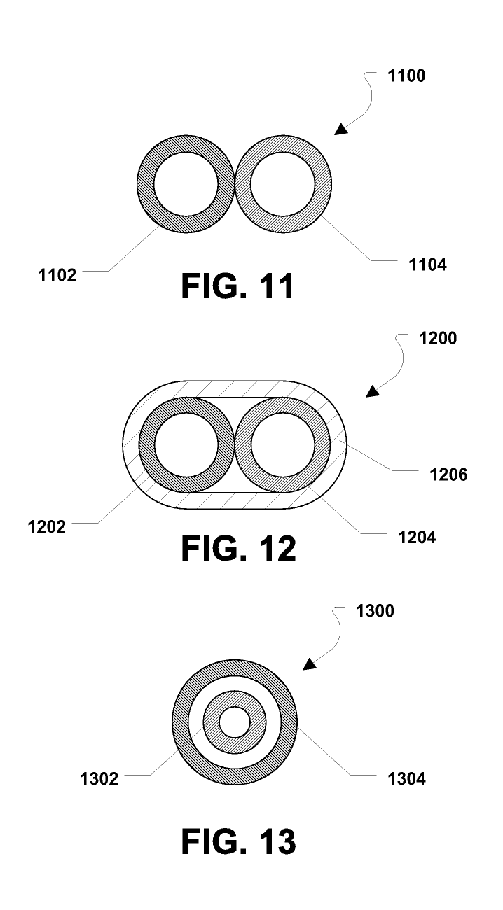

FIG. 11 illustrates a first embodiment of an injection tube set 1100 that can be used in conjunction with the first nucleus implant 500 or second nucleus implant 800, described above. As shown, the injection tube set 1100 includes a first injection tube 1102 and second injection tube 1104. Further, the injection tubes 1102, 1104 are separate tubes.

FIG. 12 shows a second embodiment of an injection tube set 1200 that can be used in conjunction with the first nucleus implant 500 or second nucleus implant 800, described above. As shown, the injection tube set 1200 can include a first injection tube 1202 and second injection tube 1204. Further, the injection tubes 1202, 1204 can be positioned tangential to each other and the injection tubes 1202, 1204 can be disposed, or otherwise placed, within a jacket 1206. In a particular embodiment, the jacket 1206 can protect the injection tubes 1202, 1204 and the jacket 1206 can facilitate insertion of a nucleus implant within an intervertebral disc.

FIG. 13 depicts a third embodiment of an injection tube set 1300 that can be used in conjunction with the first nucleus implant 500 or second nucleus implant 800, described above. As shown, the injection tube set 1300 includes a first injection tube 1302 and second injection tube 1304 disposed around the first injection tube 1302. In this particular embodiment, the first injection tube 1302 and the second injection tube 1304 can be coaxial or concentric.

### DESCRIPTION OF A FIRST EMBODIMENT OF A METHOD OF INSTALLING A NUCLEUS IMPLANT

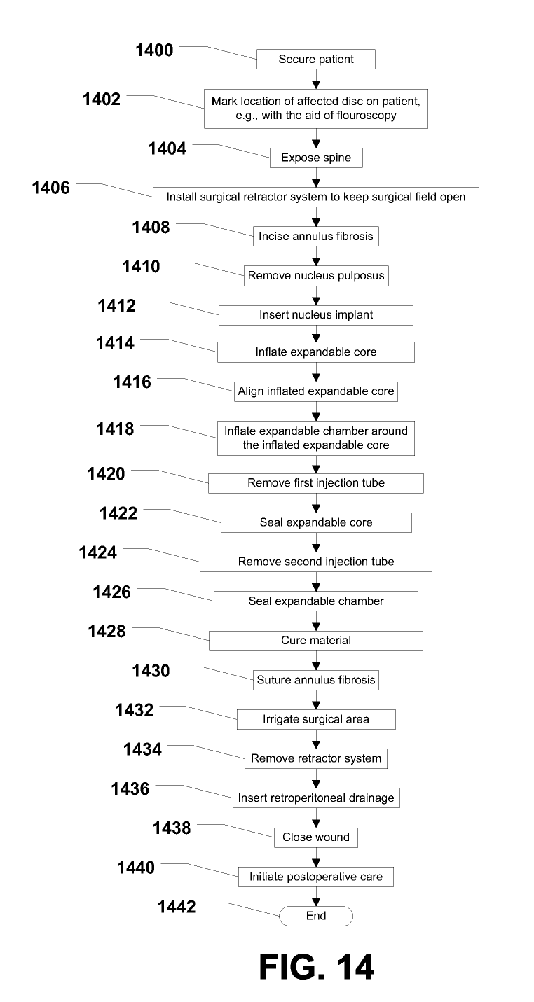

Referring to FIG. 14, an exemplary, non-limiting embodiment of a method of installing a nucleus implant is shown and commences at block 1400. At block 1400, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 1402, the location of the affected disc is marked on the patient, e.g., with the aid of fluoroscopy. At block 1404, the surgical area along spinal column is exposed. Further, at block 1406, a surgical retractor system can be installed to keep the surgical field open. For example, the surgical retractor system can be a Medtronic Sofamor Danek Endoring™ Surgical Retractor System.

Proceeding to block 1408, the annulus fibrosus of the affected disc is incised to expose the nucleus pulposus. Further, at block 1410, the nucleus pulposus is removed to create an intervertebral disc space within the annulus fibrosus. At block 1412, the nucleus implant is inserted within the intervertebral disc space of the annulus fibrosus. Further, at block 1414, the expandable core is inflated. At block 1416, the inflated core is aligned. Moving to block 1418, the expandable chamber is inflated, or otherwise expanded, around the inflated core, thereby enabling positioning and retention of the core. Alternatively, the chamber may be inflated before the core.

At block 1420, the first injection tube, i.e., the injection tube attached to the expandable core, can be removed. Continuing to block 1422, the expandable core is sealed—if the expandable chamber is not self-sealing, e.g., with a self-sealing valve. At block 1424, the second injection tube, i.e., the injection tube coupled to the expandable chamber, can be removed. Moreover, at block 1426, the expandable chamber is sealed—if the expandable chamber is not self-sealing, e.g., with a self-sealing valve. At block 1428, the material used to inflate, or expand, the expandable core and the expandable chamber can be cured. In a particular embodiment, the material can be allowed to cure naturally under the ambient conditions of the operating room. Alternatively, the material can be cured using an energy source. For example, the energy source can be a light source that emits visible light, infrared (IR) light, or ultra-violet (UV) light. Further, the energy source can be a heating device, a radiation device, or other mechanical device.

Proceeding to block 1430, the annulus fibrosus is sutured. At block 1432, the intervertebral space can be irrigated. Further, at block 1434, the retractor system can be removed. At block 1436, a drainage, e.g., a retroperitoneal drainage, can be inserted into the wound. Additionally, at block 1438, the surgical wound can be closed. The surgical wound can be closed using sutures, surgical staples, or any other surgical technique well known in the art. Moving to block 1440, postoperative care can be initiated. The method ends at state 1442.

### DESCRIPTION OF A THIRD EMBODIMENT OF A NUCLEUS IMPLANT

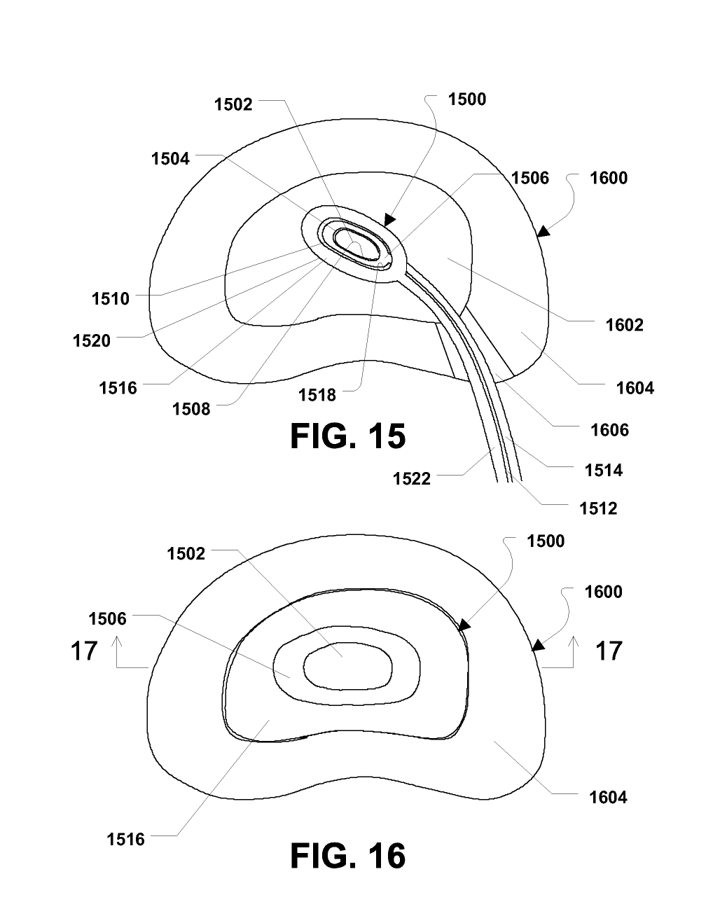

Referring to FIG. 15 through FIG. 17, a third embodiment of a nucleus implant is shown and is designated 1500. As shown, the nucleus implant 1500 includes an expandable core 1502 that defines an outer surface 1504. In a particular embodiment, when inflated, the expandable core 1502 can have a cross-section that is generally elliptical. Alternatively, when inflated, the expandable core 1502 can have a cross-section that is: generally circular, generally rectangular, generally square, generally triangular, generally trapezoidal, generally rhombic, generally quadrilateral, any generally polygonal shape, or any combination thereof.

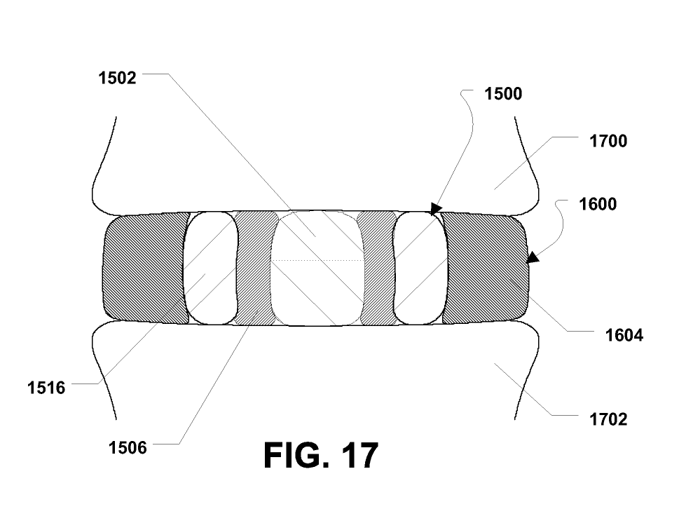

As illustrated in FIG. 15 and FIG. 16, a first expandable chamber 1506 can be disposed around the expandable core 1502. In a particular embodiment, as shown, the first expandable chamber 1506 can have a generally toroidal shape. Further, as shown in FIG. 17, the first expandable chamber 1506 can have a cross-section that is generally shaped like a kidney bean when the first expandable chamber 1506 is inflated. Alternatively, when inflated, the first expandable chamber 1506 can have a cross-section that is: generally elliptical, generally circular, generally rectangular, generally square, generally triangular, generally trapezoidal, generally rhombic, generally quadrilateral, any generally polygonal shape, or any combination thereof.

The first expandable chamber 1506 can define an inner surface 1508 and an outer surface 1510. In a particular embodiment, the inner surface 1508 of the first expandable chamber 1506 can be attached to the outer surface 1504 of the expandable core 1502. As such, proper placement of the first expandable chamber 1506 can be based on the placement of the expandable core 1502. Alternatively, the first expandable chamber 1506 can be separate from the expandable core 1502 and the first expandable chamber 1506 may engage the expandable core 1502 after the first expandable chamber 1506 is properly inflated.

As depicted in FIG. 15, the nucleus implant 1500 can include a first injection tube 1512 that extends from the outer surface 1504 of the expandable core 1502. Further, the nucleus implant 1500 can include a second injection tube 1514 that extends from the outer surface 1510 of the first expandable chamber 1506.

FIG. 15 through FIG. 17 further show that the nucleus implant 1500 can include a second expandable chamber 1516 that can be disposed around the first expandable chamber 1506. In a particular embodiment, the second expandable chamber 1516 can have a generally toroidal shape. Further, when inflated, the second expandable chamber 1516 can have a cross-section that is generally shaped like a kidney bean. Alternatively, when inflated, the second expandable chamber 1516 can have a cross-section that is: generally elliptical, generally circular, generally rectangular, generally square, generally triangular, generally trapezoidal, generally rhombic, generally quadrilateral, any generally polygonal shape, or any combination thereof.

The second expandable chamber 1516 can define an inner surface 1518 and an outer surface 1520. In a particular embodiment, the inner surface 1518 of the second expandable chamber 1516 can be attached to the outer surface 1510 of the first expandable chamber 1506 and the inner surface 1508 of the first expandable chamber 1506 can be attached to the outer surface 1504 of the expandable core 1502. Alternatively, the second expandable chamber 1516 can be separate from the first expandable chamber 1506 and the expandable core 1502. In such a configuration, the second expandable chamber 1516 can engage the first expandable chamber 1506 after the first expandable chamber 1506 and the second expandable chamber 1516 are properly inflated. An implant with several chambers may enable more fine adjustment of the position of the core than with a single chamber.

As illustrated in FIG. 15, the nucleus implant 1500 can include a third injection tube 1522 that extends from the outer surface 1520 of the second expandable chamber 1516. In a particular embodiment, each of the expandable core 1502, the first expandable chamber 1506, and the second expandable chamber 1516 of the nucleus implant 1500 is expandable from a deflated position, shown in FIG. 15, to one selected position among a plurality of inflated positions, shown in FIG. 16, up to a maximum inflated position. Further, after the expandable core 1502, the first expandable chamber 1506, and the second expandable chamber 1516 are inflated, or otherwise expanded, the injection tubes 1512, 1514, 1522 can be removed, as depicted in FIG. 16.

In a particular embodiment, the nucleus implant 1500 can include a first self-sealing valve (not shown) within the outer surface 1504 of the expandable core 1502, e.g., adjacent to the first injection tube 1512. The nucleus implant 1500 can also include a second self-sealing valve (not shown) within the outer surface 1510 of the first expandable chamber 1506, e.g., adjacent to the second injection tube 1514. Further, the nucleus implant 1500 can include a third self-sealing valve (not shown) within the outer surface 1520 of the second expandable chamber 1516, e.g., adjacent to the third injection tube 1522. The self-sealing valves can prevent the expandable core 1502 and the expandable chambers 1506, 1516 from leaking material after the expandable core 1502 and the expandable chambers 1506, 1516 are inflated and the injection tubes 1512, 1514, 1522 are removed.

FIG. 17 indicates that the nucleus implant 1500 can be implanted within an intervertebral disc 1600 between a superior vertebra 1700 and an inferior vertebra 1702. More specifically, the nucleus implant 1500 can be implanted within an intervertebral disc space 1602 established within the annulus fibrosus 1604 of the intervertebral disc 1600. The intervertebral disc space 1602 can be established by removing the nucleus pulposus (not shown) from within the annulus fibrosus 1604.

In a particular embodiment, the expandable core 1502, the first expandable chamber 1506, and the second expandable chamber 1516 can be inflated so the inner surface 1508 of the first expandable chamber 1506 engages the outer surface of the expandable core 1502 and the outer surface 1510 of the first expandable chamber 1506 engages the inner surface 1518 of the second expandable chamber 1516. Further, the outer surface 1520 of the second expandable chamber 1516 can engage the annulus fibrosis 1604.

The nucleus implant 1500 can provide shock-absorbing characteristics substantially similar to the shock absorbing characteristics provided by the nucleus pulposus. Further, in a particular embodiment, the hardness of the material used to inflate the expandable core 1502 of the nucleus implant 1500 is greater than or equal to the hardness of the material used to inflate the first expandable chamber 1506, i.e., after the materials cure. Moreover, the hardness of the material used to inflate the first expandable chamber 1506 can be greater than or equal to the hardness of the material used to inflate the second expandable chamber 1516, e.g., after those materials cure.

Arranging several expandable chambers around a core may result in an implant with hardness that varies more progressively from the core towards the periphery than with a single chamber. Thus, an implant with a very hard core and a very soft periphery may be obtained. Moreover, an implant with several variable hardness chambers may more easily spread the loads exerted at the vertebral level. In addition, the mobility of the thus arranged implant is better controlled. As one example, the core has a hardness of 55 Shore D, the first chamber has a hardness of 50 Shore D and the second chamber has a hardness of 40 Shore D.

Alternatively, the viscosity of the material used to inflate the expandable core 1502 of the nucleus implant 1500 can be greater than or equal to the viscosity of the material used to inflate the first expandable chamber 1506. Also, the viscosity of the material used to inflate the first expandable chamber 1506 can be greater than or equal to the viscosity of the material used to inflate the second expandable chamber 1516.

Additionally, the height of the expandable core 1502, when expanded, can be greater than or equal to the height of the first expandable chamber 1506 when expanded. Also, the height of the first expandable chamber 1506, when expanded, can be greater than or equal to the height of the second expandable chamber 1516 when expanded. As shown in FIG. 17, the expandable core 1502, the first expandable chamber 1506, and the second expandable chamber 1516 of the nucleus implant 1500 can be configured to provide proper support and spacing between the superior vertebra 1700 and the inferior vertebra 1702.

In a particular embodiment, the expandable core 1502, the first expandable chamber 1506, the second expandable chamber 1516, or a combination of the expandable core 1502, the first expandable chamber 1506, and the second expandable chamber 1516 of the nucleus implant 1500 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the expandable core 1502, the first expandable chamber 1506, the second expandable chamber 1516, or a combination of the expandable core 1502, the first expandable chamber 1506, and the second expandable chamber 1516 of the nucleus implant 1500 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 1500 can be installed using a posterior surgical approach, as shown. Further, the nucleus implant 1500 can be installed through a posterior incision 1606 made within the annulus fibrosus 1604 of the intervertebral disc 1600. Alternatively, the nucleus implant 1500 can be installed using an anterior surgical approach, a lateral surgical approach, or any other surgical approach well known in the art.

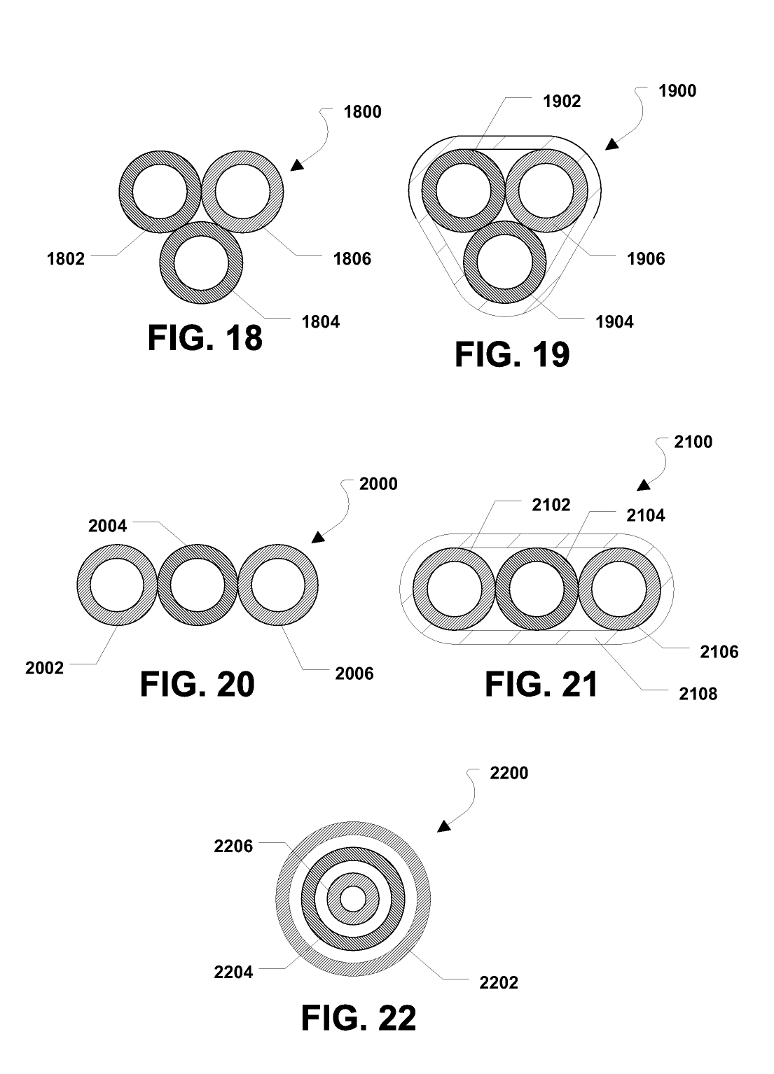

FIG. 18 illustrates a first embodiment of an injection tube set 1800 that can be used in conjunction a nucleus implant, e.g., the third nucleus implant 1500 described herein. As shown, the injection tube set 1800 includes a first injection tube 1802, a second injection tube 1804, and a third injection tube 1806. As shown, each injection tube 1802, 1804, 1806 can be tangentially connected to two other injection tubes such that a cross-section of the injection tube set 1800 is generally triangular.

FIG. 19 shows a second embodiment of an injection tube set 1900 that can be used in conjunction with a nucleus implant, e.g., the third nucleus implant 1500 described above. As shown, the injection tube set 1900 can include a first injection tube 1902, a second injection tube 1904, and a third injection tube 1906. Each injection tube 1902, 1904, 1906 can be tangentially connected to two other injection tubes such that a cross-section of the injection tube set 1900 is generally triangular. Further, in a particular embodiment, a jacket 1908 can be disposed around the injection tubes 1902, 1904, 1906 along the length of the injection tubes 1902, 1904, 1906. The jacket 1908 can protect the injection tubes 1902, 1904, 1906 and the jacket 1908 can facilitate insertion of a nucleus implant within an intervertebral disc.

FIG. 20 illustrates a third embodiment of an injection tube set 2000 that can be used in conjunction a nucleus implant, e.g., the third nucleus implant 1500 described herein. As shown, the injection tube set 2000 includes a first injection tube 2002, a second injection tube 2004, and a third injection tube 2006. As shown, the first injection tube 2002 and the third injection tube 2006 can be tangentially connected to the second injection tube 2004 such that the cross-section of the injection tube set 2000 is generally flat.

FIG. 21 illustrates a fourth embodiment of an injection tube set 2100 that can be used in conjunction a nucleus implant, e.g., the third nucleus implant 1500 described herein. As shown, the injection tube set 2100 includes a first injection tube 2102, a second injection tube 2104, and a third injection tube 2106. As shown, the first injection tube 2102 and the third injection tube 2106 can be tangentially connected to the second injection tube 2104 such that the cross-section of the injection tube set 2100 is generally flat. Further, in a particular embodiment, a jacket 2108 can be disposed around the injection tubes 2102, 2104, 2106 along the length of the injection tubes 2102, 2104, 2106. The jacket 2108 can protect the injection tubes 2102, 2104, 2106 and the jacket 2108 can facilitate insertion of a nucleus implant within an intervertebral disc.

FIG. 22 depicts a fifth embodiment of an injection tube set 2200 that can be used in conjunction with a nucleus implant, e.g., the third nucleus implant 1500 described above. As shown, the injection tube set 2200 includes a first injection tube 2206. A second injection tube 2204 can be disposed around the first injection tube 2206. Further, a third injection tube 2202 can be disposed around the second injection tube 2204. In this particular embodiment, the first injection tube 2206, the second injection tube 2204, and the third injection tube 2202 can be coaxial or concentric.

### DESCRIPTION OF A SECOND EMBODIMENT OF A METHOD OF INSTALLING A NUCLEUS IMPLANT

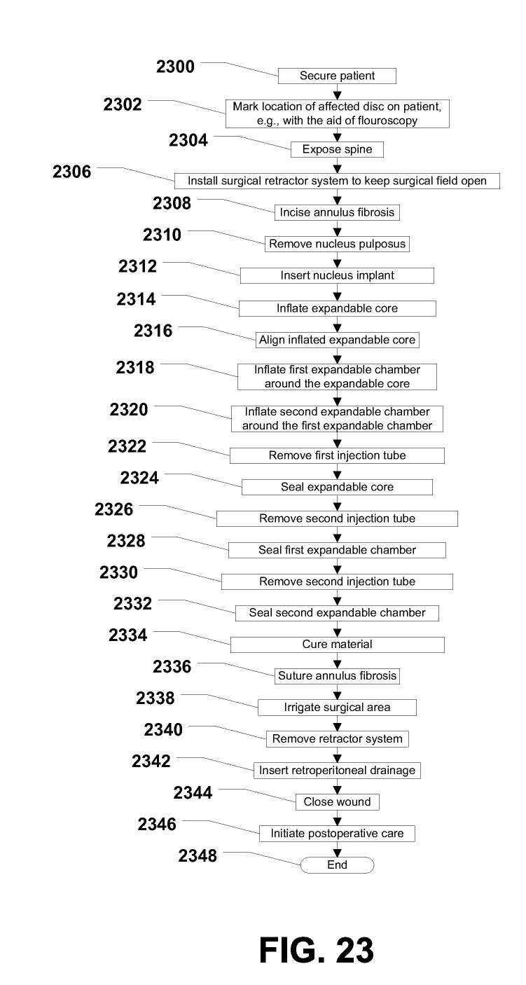

Referring to FIG. 23, a second exemplary, non-limiting embodiment of a method of installing a nucleus implant is shown and commences at block 2300. At block 2300, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 2302, the location of the affected disc is marked on the patient, e.g., with the aid of fluoroscopy. At block 2304, the surgical area along spinal column is exposed. Further, at block 2306, a surgical retractor system can be installed to keep the surgical field open. For example, the surgical retractor system can be a Medtronic Sofamor Danek Endoring™ Surgical Retractor System.

Proceeding to block 2308, the annulus fibrosus of the affected disc is incised to expose the nucleus pulposus. Further, at block 2310, the nucleus pulposus is removed to create an intervertebral disc space within the annulus fibrosus. At block 2312, the nucleus implant is inserted within the intervertebral disc space of the annulus fibrosus. Further, at block 2314, the expandable core is inflated. At block 2316, the inflated core is aligned. Moving to block 2318, the first expandable chamber is inflated, or otherwise expanded, around the inflated core. At block 2320, the second expandable chamber is inflated, or otherwise inflated, around the first expandable chamber.

Proceeding to block 2322, the first injection tube, i.e., the injection tube attached to the expandable core, can be removed. At block 2324, the expandable core is sealed—if the expandable chamber is not self-sealing, e.g., with a self-sealing valve. At block 2326, the second injection tube, i.e., the injection tube coupled to the first expandable chamber, can be removed. Moreover, at block 2328, the first expandable chamber is sealed—if the first expandable chamber is not self-sealing, e.g., with a self-sealing valve. Further, at block 2330, the third injection tube, i.e., the injection tube coupled to the second expandable chamber, can be removed. Moreover, at block 2332, the second expandable chamber is sealed—if the second expandable chamber is not self-sealing, e.g., with a self-sealing valve. At block 2334, the material used to inflate, or expand, the expandable core and the expandable chambers can be cured. In a particular embodiment, the material can be allowed to cure naturally under the ambient conditions of the operating room. Alternatively, the material can be cured using an energy source. For example, the energy source can be a light source that emits visible light, infrared (IR) light, or ultra-violet (UV) light. Further, the energy source can be a heating device, a radiation device, or other mechanical device.

Proceeding to block 2336, the annulus fibrosus is sutured. At block 2338, the intervertebral space can be irrigated. Further, at block 2340, the retractor system can be removed. At block 2342, a drainage, e.g., a retroperitoneal drainage, can be inserted into the wound. Additionally, at block 2344, the surgical wound can be closed. The surgical wound can be closed using sutures, surgical staples, or any other surgical technique well known in the art. Moving to block 2346, postoperative care can be initiated. The method ends at state 2348.

### DESCRIPTION OF A FOURTH EMBODIMENT OF A NUCLEUS IMPLANT

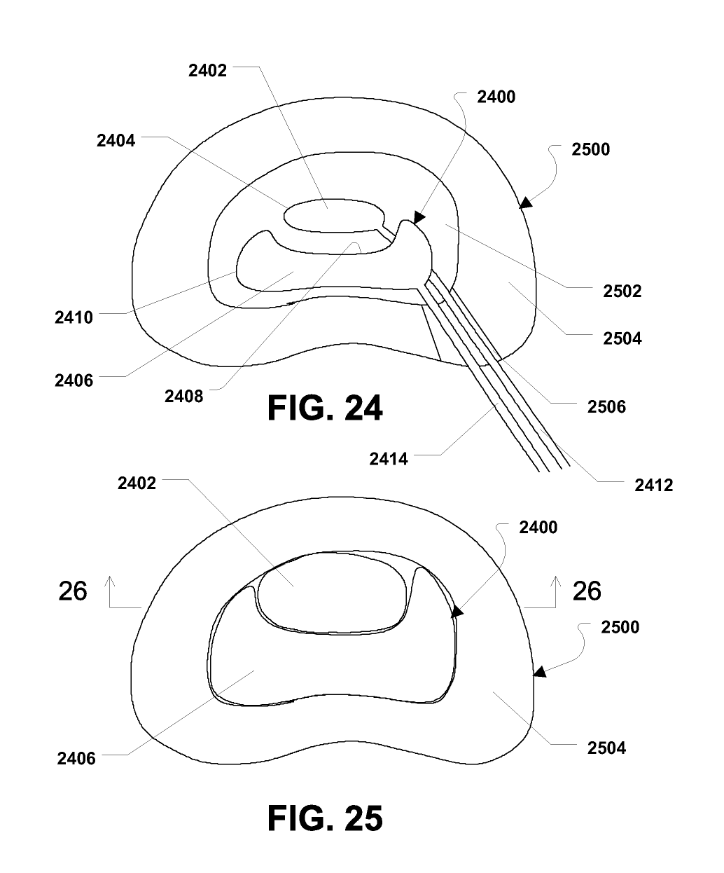

Referring to FIG. 24 through FIG. 26, an embodiment of a nucleus implant is shown and is designated 2400. As shown, the nucleus implant 2400 includes an expandable core 2402 that defines an outer surface 2404. In a particular embodiment, when inflated, the expandable core 2402 can have a cross-section that is generally elliptical. Alternatively, when inflated, the expandable core 2402 can have a cross-section that is: generally circular, generally rectangular, generally square, generally triangular, generally trapezoidal, generally rhombic, generally quadrilateral, any generally polygonal shape, or any combination thereof.

As illustrated in FIG. 24 through FIG. 26, an expandable chamber 2406 can be disposed around the expandable core 2402. In a particular embodiment, as shown, the expandable chamber 2406 can be generally shaped like the letter “U” and the expandable chamber 2406 can be inflated, or otherwise expanded, around the expandable core 2402.

The U-shaped chamber is particularly suited for avoiding the migration of the core towards the incision through which it has been inserted. This is because the U shape partially surrounding the core blocks this incision. This U shape is also advantageous when the intervertebral disc shape has, in a saggital plane, an obvious trapezoidal shape. An intermediate expandable chamber occupying the space between the core 2402 and the U chamber 2406 (FIG. 24) may be envisaged. This additional arrangement may allow more accurate positioning of the core.

The expandable chamber 2406 can define a first surface 2408 and a second surface 2410. In a particular embodiment, the first surface 2408 of the expandable chamber 2406 can be attached to the outer surface 2404 of the expandable core 2402. As such, proper placement of the expandable chamber 2406 can be based on the placement of the expandable core 2402. Alternatively, the expandable chamber 2406 can be separate from the expandable core 2402 and the expandable chamber 2406 may engage the expandable core 2402 after the expandable chamber 2406 is properly inflated.

As depicted in FIG. 24, the nucleus implant 2400 can include a first injection tube 2412 that extends from the outer surface 2404 of the expandable core 2402. Further, the nucleus implant 2400 can include a second injection tube 2414 that extends from the second surface 2410 of the expandable chamber 2406. In a particular embodiment, each of the expandable core 2402 and the expandable chamber 2406 of the nucleus implant 2400 is expandable from a deflated position, shown in FIG. 24, to one selected position among a plurality of inflated positions, shown in FIG. 25, up to a maximum inflated position. Further, after the expandable core 2402 and the expandable chamber 2406 are inflated, or otherwise expanded, the injection tubes 2412, 2414 can be removed, as depicted in FIG. 25.

In a particular embodiment, the nucleus implant 2400 can include a first self-sealing valve (not shown) within the outer surface 2404 of the expandable core 2402, e.g., adjacent to the first injection tube 2412. Also, the nucleus implant 2400 can include a second self-sealing valve (not shown) within the second surface 2410 of the expandable chamber 2406, e.g., adjacent to the second injection tube 2414. The self-sealing valves can prevent the expandable core 2402 and the expandable chamber 2406 from leaking material after the expandable core 2402 and the expandable chamber 2406 are inflated and the injection tubes 2412, 2414 is removed.

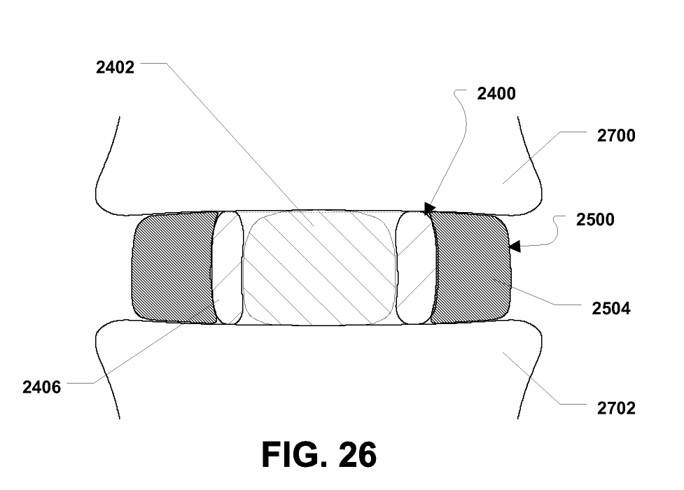

FIG. 26 indicates that the nucleus implant 2400 can be implanted within an intervertebral disc 2500 between a superior vertebra 2600 and an inferior vertebra 2602. More specifically, the nucleus implant 2400 can be implanted within an intervertebral disc space 2502 established within the annulus fibrosus 2504 of the intervertebral disc 2500. The intervertebral disc space 2502 can be established by removing the nucleus pulposus (not shown) from within the annulus fibrosus 2504.

In a particular embodiment, the expandable core 2402 and the expandable chamber 2406 can be inflated so the first surface 2408 of the expandable chamber 2406 engages a portion of the outer surface of the expandable core 2402 and the second surface 2410 of the expandable chamber 2406 engages a portion of the annulus fibrosis 2504. Further, portions of the outer surface 2410 of the expandable chamber 2406 can engage the superior vertebra 2600 and an inferior vertebra 2602. Moreover, when the expandable chamber 2406 is expanded, or otherwise inflated, the expandable chamber 2406 at least partially surrounds the expandable core 2402. As depicted in FIG. 25, the core 2402 is placed between the arms of the U formed by the chamber 2406.

The nucleus implant 2400 can provide shock-absorbing characteristics substantially similar to the shock absorbing characteristics provided by the nucleus pulposus. Further, in a particular embodiment, the hardness of the material used to inflate the expandable core 2402 of the nucleus implant 2400 is greater than or equal to the hardness of the material used to inflate the expandable chamber 2406, i.e., after the materials cure. Alternatively, the viscosity of the material used to inflate the expandable core 2402 is greater than or equal to the viscosity of the material used to inflate the expandable chamber 2406.

Also, the overall height of the expandable core 2402 is greater than or equal to the overall height of the expandable chamber 2406 when inflated. As shown in FIG. 26, the expandable core 2402 and the expandable chamber 2406 of the nucleus implant 2400 can be configured to provide proper support and spacing between the superior vertebra 2600 and the inferior vertebra 2602.

In a particular embodiment, the expandable core 2402, the expandable chamber 2406, or a combination of the expandable core 2402 and the expandable chamber 2406 of the nucleus implant 2400 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the expandable core 2402, the expandable chamber 2406, or a combination of the expandable core 2402 and the expandable chamber 2406 of the nucleus implant 2400 can be inflated with one or more of the following: fibroblasts, lipoblasts, chondroblasts, differentiated stem cells or other biologic factor which would create a motion limiting tissue when injected into a bioresorbable motion limiting scaffold.

The material or materials used for generating the expansion of the core and the chamber can be different for the core and the chamber. This holds true for this or any of the embodiments described herein, particularly when the implant comprises a core and several chambers.

In a particular embodiment, the nucleus implant 2400 can be installed using a posterior surgical approach, as shown. Further, the nucleus implant 2400 can be installed through a posterior incision 2506 made within the annulus fibrosus 2504 of the intervertebral disc 2500. Alternatively, the nucleus implant 2400 can be installed using an anterior surgical approach, a lateral surgical approach, or any other surgical approach well known in the art.

### DESCRIPTION OF A THIRD EMBODIMENT OF A METHOD OF INSTALLING A NUCLEUS IMPLANT

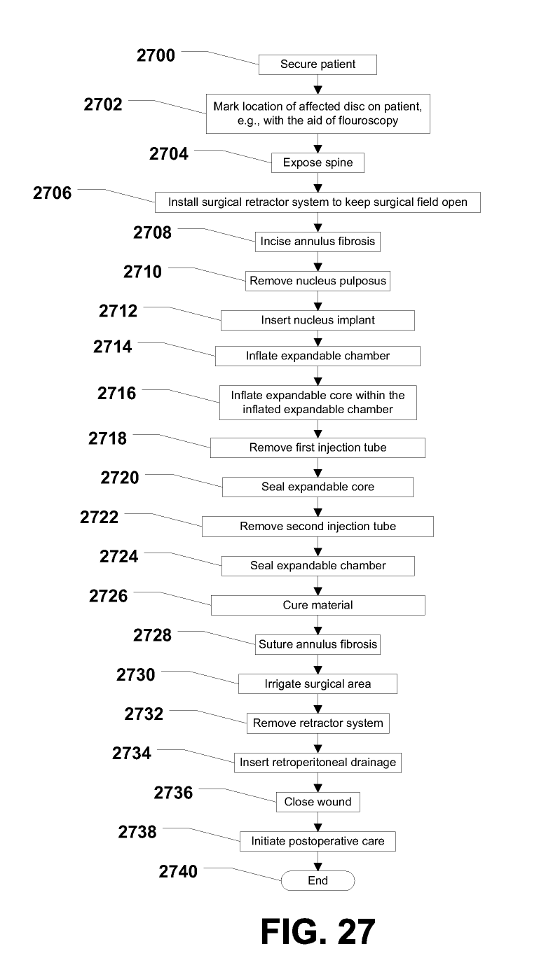

Referring to FIG. 27, an exemplary, non-limiting embodiment of a method of installing a nucleus implant is shown and commences at block 2700. At block 2700, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 2702, the location of the affected disc is marked on the patient, e.g., with the aid of fluoroscopy. At block 2704, the surgical area along spinal column is exposed. Further, at block 2706, a surgical retractor system can be installed to keep the surgical field open. For example, the surgical retractor system can be a Medtronic Sofamor Danek Endoring™ Surgical Retractor System.

Proceeding to block 2708, the annulus fibrosus of the affected disc is incised to expose the nucleus pulposus. Further, at block 2710, the nucleus pulposus is removed to create an intervertebral disc space within the annulus fibrosus. At block 2712, the nucleus implant is inserted within the intervertebral disc space of the annulus fibrosus. Further, at block 2714, the expandable chamber is inflated. Moving to block 2716, the expandable core is inflated, or otherwise expanded, within the inflated expandable chamber.

At block 2718, the first injection tube, i.e., the injection tube attached to the expandable core, can be removed. Continuing to block 2720, the expandable core is sealed—if the expandable chamber is not self-sealing, e.g., with a self-sealing valve. At block 2722, the second injection tube, i.e., the injection tube coupled to the expandable chamber, can be removed. Moreover, at block 2724, the expandable chamber is sealed—if the expandable chamber is not self-sealing, e.g., with a self-sealing valve. At block 2726, the material used to inflate, or expand, the expandable core and the expandable chamber can be cured. In a particular embodiment, the material can be allowed to cure naturally under the ambient conditions of the operating room. Alternatively, the material can be cured using an energy source. For example, the energy source can be a light source that emits visible light, infrared (IR) light, or ultra-violet (UV) light. Further, the energy source can be a heating device, a radiation device, or other mechanical device.

Proceeding to block 2728, the annulus fibrosus is sutured. At block 2730, the intervertebral space can be irrigated. Further, at block 2732, the retractor system can be removed. At block 2734, a drainage, e.g., a retroperitoneal drainage, can be inserted into the wound. Additionally, at block 2736, the surgical wound can be closed. The surgical wound can be closed using sutures, surgical staples, or any other surgical technique well known in the art. Moving to block 2738, postoperative care can be initiated. The method ends at state 2740.

### CONCLUSION

With the configuration of structure described above, the nucleus implant according to one or more of the embodiments provides a device that may be implanted to replace the nucleus pulposus within a natural intervertebral disc that is diseased, degenerated, or otherwise damaged. The nucleus implant can be disposed within an intervertebral disc space that can be established within an intervertebral disc by removing the nucleus pulposus.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A nucleus implant configured to be installed within an intervertebral disc between an inferior vertebra and a superior vertebra, the nucleus implant comprising:
an expandable core; and
an expandable chamber disposed at least partially around the expandable core, wherein the expandable chamber is expandable from a deflated position to an inflated position, and wherein a hardness of the expandable core when inflated is greater than or equal to a hardness of the expandable chamber when inflated.

2. The nucleus implant of claim 1, wherein the expandable chamber is configured to engage an annulus fibrosus when inflated.

3. The nucleus implant of claim 2, wherein the expandable core, the expandable chamber, or a combination thereof is configured to engage the superior vertebra and the inferior vertebra when inflated.

4. The nucleus implant of claim 1, wherein a height of the expandable core is greater than or equal to a height of the expandable chamber when the expandable chamber is inflated.

5. The nucleus implant of claim 2, wherein the expandable core, the expandable chamber, or a combination thereof is inflated with an injectable extended use approved medical material.

6. The nucleus implant of claim 5, wherein the injectable extended use approved medical material comprises a polymer material.

7. The nucleus implant of claim 6, wherein the polymer material comprises a polyurethane material, a polyolefin material, a polyether material, a silicone material, or a combination thereof.

8. The nucleus implant of claim 7, wherein the polyolefin material comprises a polypropylene, polyethylene, halogenated polyolefin, flouropolyolefin, or a combination thereof.

9. The nucleus implant of claim 7, wherein the polyether material comprises polyetherketone (PEK), polyetheretherketone (PEEK), polyetherketoneketone (PEKK), polyaryletherketone (PAEK), or a combination thereof.

10. The nucleus implant of claim 7, wherein the silicon material comprises silicone hydrogel.

11. The nucleus implant of claim 5, wherein the injectable extended use approved medical material comprises sterile water, saline, sterile air, or a combination thereof.

12. The nucleus implant of claim 1, further comprising a first injection tube extending from the expandable core wherein the first injection tube is configured to be removed after the nucleus implant is installed within an intervertebral disc.

13. The nucleus implant of claim 12, further comprising a second injection tube extending from the expandable chamber, wherein the second injection tube is configured to be removed after the nucleus implant is installed within an intervertebral disc.

14. The nucleus implant of claim 1, wherein the expandable chamber is generally toroid shaped and includes an inner surface and wherein the inner surface of the expandable chamber engages an outer surface of the expandable core when the expandable core and the expandable chamber are inflated.

15. The nucleus implant of claim 14, wherein the inner surface of the expandable chamber is attached to the outer surface of the expandable core.

16. The nucleus implant of claim 1, wherein the expandable chamber is a first expandable chamber and wherein the nucleus implant further comprises a second expandable chamber at least partially around the first expandable chamber.

17. The nucleus implant of claim 16, wherein the second expandable chamber is expandable from a deflated position to an inflated position.

18. The nucleus implant of claim 18, wherein the second expandable chamber is configured to engage an annulus fibrosus when inflated.

19. The nucleus implant of claim 18, wherein the expandable core, the first expandable chamber, the second expandable chamber, or a combination thereof is configured to engage the superior vertebra and the inferior vertebra when inflated.

20. The nucleus implant of claim 1, wherein the expandable chamber is generally shaped like an inverted bowl and wherein the expandable chamber is disposed over the expandable core.

21. The nucleus implant of claim 1, wherein the expandable chamber engages an annulus fibrosis and engages the superior vertebra and the inferior vertebra.

22. The nucleus implant of claim 21, wherein a portion of the expandable chamber is disposed between the expandable core and the superior vertebra.

23. The nucleus implant of claim 1, wherein the expandable chamber is generally shaped like the letter U.

24. The nucleus implant of claim 23, wherein the expandable chamber is configured to be inflated around the expandable core.

25. The nucleus implant of claim 24, wherein the expandable core is configured to be installed in an anterior position within an intervertebral disc and wherein the expandable chamber is configured to be installed posterior to the expandable core.

26. A nucleus implant configured to be installed within an intervertebral disc between an inferior vertebra and a superior vertebra, the nucleus implant comprising:
an expandable core; and
a toroid shaped expandable chamber disposed at least partially around the expandable core, wherein a hardness of the expandable core when inflated is greater than or equal to a hardness of the toroid shaped expandable chamber when inflated.

27. The nucleus implant of claim 26, wherein the toroid shaped expandable chamber includes an inner surface and an outer surface, wherein the expandable core includes an outer surface, and wherein the inner surface of the toroid shaped expandable chamber is configured to engage the outer surface of the expandable core when the toroid shaped expandable chamber and the expandable core are inflated.

28. The nucleus implant of claim 27, wherein the expandable chamber is configured to engage an annulus fibrosus of the intervertebral disc when inflated.

29. The nucleus implant of claim 28, wherein the expandable core, the expandable chamber, or a combination thereof is configured to engage the superior vertebra and the inferior vertebra when inflated.

30. The nucleus implant of claim 29, further comprising a first injection tube extending from the expandable core wherein the first injection tube is configured to be removed after the nucleus implant is installed.

31. The nucleus implant of claim 30, further comprising a second injection tube extending from the toroid shaped expandable chamber, wherein the second injection tube is configured to be removed after the toroid shaped expandable chamber is inflated.

32. The nucleus implant of claim 26, wherein a height of the expandable core is greater than or equal to a height of the expandable chamber when the expandable chamber is inflated.

33. The nucleus implant of claim 26, wherein the toroid shaped expandable chamber is attached to the expandable core.

34. The nucleus implant of claim 33, wherein the inner surface of the toroid shaped expandable chamber is attached to the outer surface of the expandable core.

35. A nucleus implant configured to be installed within an intervertebral disc between an inferior vertebra and a superior vertebra, the nucleus implant comprising:
an expandable core;
a first toroid shaped expandable chamber disposed at least partially around the expandable core; and
a second toroid shaped expandable chamber disposed at least partially around the first toroid shaped expandable chamber, wherein a hardness of the expandable core is greater than or equal to a hardness of the first expandable chamber and the hardness of the first expandable chamber is greater than or equal to a hardness of the second expandable chamber when the expandable core, the first expandable chamber, and the second expandable chamber are inflated.

36. The nucleus implant of claim 35, wherein the expandable core includes an outer surface, wherein the first toroid shaped expandable chamber includes an inner surface and an outer surface, wherein the second toroid shaped expandable chamber includes an inner surface and an outer surface, and wherein the inner surface of the first toroid shaped expandable chamber is configured to engage the outer surface of the expandable core, the inner surface of the second expandable chamber engages the outer surface of the first toroid shaped expandable chamber, and the outer surface of the second toroid shaped expandable chamber is configured to engage an annulus fibrosus of the intervertebral disc when the expandable core, the first toroid shaped expandable chamber, and the second toroid shaped expandable chamber are inflated.

37. The nucleus implant of claim 36, wherein the expandable core, the first toroid shaped expandable chamber, the second toroid shaped expandable chamber, or a combination thereof is configured to engage the superior vertebra and the inferior vertebra when inflated.

38. The nucleus implant of claim 36, further comprising a first injection tube extending from the expandable core wherein the first injection tube is configured to be removed after the expandable core is inflated.

39. The nucleus implant of claim 38, further comprising a second injection tube extending from the first toroid shaped expandable chamber, wherein the second injection tube is configured to be removed after the first toroid shaped expandable chamber is inflated.

40. The nucleus implant of claim 39, further comprising a third injection tube extending from the second toroid shaped expandable chamber, wherein the third injection tube is configured to be removed after the second toroid shaped expandable chamber is inflated.

41. The nucleus implant of claim 35, wherein a height of the expandable core is greater than or equal to a height of the first toroid shaped expandable chamber and the height of the first toroid shaped expandable chamber is greater than or equal to a height of the second toroid shaped expandable chamber when the expandable core, the first toroid shaped expandable chamber, and the second toroid shaped expandable chamber are inflated.

42. The nucleus implant of claim 36, wherein the first toroid shaped expandable chamber is attached to the expandable core and wherein the second toroid shaped expandable chamber is attached to the first toroid shaped expandable chamber.

43. The nucleus implant of claim 42, wherein the inner surface of the first toroid shaped expandable chamber is attached to the outer surface of the expandable core and wherein the inner surface of the second toroid shaped expandable chamber is attached to the outer surface of the first toroid shaped expandable chamber.

44. A nucleus implant configured to be installed within an intervertebral disc between an inferior vertebra and a superior vertebra, the nucleus implant comprising:
an expandable core; and
a bowl shaped expandable chamber disposed at least partially around the expandable core, wherein a hardness of the expandable core when inflated is greater than or equal to a hardness of the bowl shaped expandable chamber when inflated.

45. The nucleus implant of clam 44, wherein the expandable core includes an outer surface and wherein the bowl shaped expandable chamber includes an inner surface and an outer surface, and wherein the inner surface of the bowl shaped expandable chamber is configured to engage the outer surface of the expandable core and wherein the outer surface of the bowl shaped expandable chamber is configured to engage an annulus fibrosus of the intervertebral disc, the superior vertebra, the inferior vertebra, or a combination thereof.

46. The nucleus implant of claim 45, wherein the expandable core, the bowl shaped expandable chamber, or a combination thereof is configured to engage the superior vertebra, the inferior vertebra, or a combination thereof when inflated.

47. The nucleus implant of claim 44, further comprising a first injection tube extending from the expandable core wherein the first injection tube is configured to assist in positioning the nucleus implant within the intervertebral disc and wherein the first injection tube is configured to be removed after the nucleus implant is installed.

48. The nucleus implant of claim 47, further comprising a second injection tube extending from the bowl shaped expandable chamber, wherein the second injection tube is configured to be removed after the bowl shaped expandable chamber is inflated.

49. The nucleus implant of claim 45, wherein the expandable chamber is attached to the expandable core.

50. The nucleus implant of claim 49, wherein the inner surface of the expandable chamber is attached to the outer surface of the expandable core.

51. A nucleus implant configured to be installed within an intervertebral disc between an inferior vertebra and a superior vertebra, the nucleus implant comprising:
an expandable core including an outer surface; and
a U shaped expandable chamber disposed at least partially around the expandable core, wherein a hardness of the expandable core when inflated is greater than or equal to a hardness of the U shaped expandable chamber when inflated.

52. The nucleus implant of claim 51, wherein the U shaped expandable chamber includes a first surface and a second surface, and wherein the first surface of the U shaped expandable chamber is configured to engage the outer surface of the expandable core and wherein the second surface of the U shaped expandable chamber is configured to engage an annulus fibrosus of the intervertebral disc.

53. The nucleus implant of claim 52, further comprising a first injection tube extending from the expandable core wherein the first injection tube is configured to assist in positioning the nucleus implant within the intervertebral disc and wherein the first injection tube is configured to be removed after the nucleus implant is installed.

54. The nucleus implant of claim 53, further comprising a second injection tube extending from the U shaped expandable chamber, wherein the second injection tube is configured to be removed after the U shaped expandable chamber is inflated.

55. The nucleus implant of claim 51, wherein a height of the expandable core is greater than or equal to a height of the U shaped expandable chamber when the U shaped expandable chamber is inflated.

56. The nucleus implant of claim 52, wherein the U shaped expandable chamber is attached to the expandable core.

57. The nucleus implant of claim 56, wherein the first surface of the U shaped expandable chamber is attached to the outer surface of the expandable core.

58. The nucleus implant of claim 51, wherein the expandable core is configured to be installed in an anterior position within the intervertebral disc and wherein the U shaped expandable chamber is configured to be installed at least partially posterior to the expandable core.

59. A method of installing a nucleus implant within an intervertebral disc between an inferior vertebra and a superior vertebra of a patient, the method comprising:
implanting the nucleus implant within the intervertebral disc, wherein the nucleus implant includes an expandable core and an expandable chamber at least partially around the expandable core; and
inflating the expandable core;
inflating the expandable chamber around the expandable core, wherein a hardness of the expandable core when inflated is greater than or equal to a hardness of the expandable chamber when inflated.

60. The method of claim 59, wherein the expandable core, the expandable chamber, or a combination thereof is configured to engage an annulus fibrosus, the superior vertebra, the inferior vertebra, or a combination thereof when the expandable core and the expandable chamber are inflated.

61. The method of claim 60, further comprising removing a first injection tube from the expandable core.

62. The method of claim 61, further comprising removing a second injection tube from the expandable chamber.

63. The method of claim 62, further comprising sealing the expandable core.

64. The method of claim 63, further comprising sealing the expandable chamber.

65. The method of claim 64, further comprising curing a material used to inflate the expandable core, the expandable chamber, or a combination thereof.

66. The method of claim 65, wherein the expandable core, the expandable chamber, or a combination thereof is inflated using an injectable extended use approved medical material.

67. The method of claim 66, wherein the injectable extended use approved medical material is a polymer material.

68. The method of claim 67, wherein the polymer material is a polyurethane material, a polyolefin material, a polyether material, a silicone material, or a combination thereof.

69. The method of claim 68, wherein the polyolefin material is a polypropylene, polyethylene, halogenated polyolefin, flouropolyolefin, or a combination thereof.

70. The method of claim 68, wherein the polyether material is polyetherketone (PEK), polyetheretherketone (PEEK), polyetherketoneketone (PEKK), polyaryletherketone (PAEK), or a combination thereof.

71. The method of claim 68, wherein the silicon material is silicone hydrogel.

72. The method of claim 66, wherein the injectable extended use approved medical material comprises sterile water, saline, sterile air, or a combination thereof.

73. A method of installing a nucleus implant within an intervertebral disc between an inferior vertebra and a superior vertebra of a patient, the method comprising:
implanting the nucleus implant within the intervertebral disc, wherein the nucleus implant includes an expandable core, a first expandable chamber at least partially around the expandable core, and a second expandable chamber at least partially around the first expandable chamber; and
inflating the expandable core;
inflating the first expandable chamber around the expandable core; and
inflating the second expandable chamber around the first expandable chamber, wherein a hardness of the expandable core when inflated is greater than or equal to a hardness of the first expandable chamber when inflated and wherein the hardness of the first expandable chamber when inflated is greater than or equal to a hardness of the second expandable chamber when inflated.

74. The method of claim 73, wherein the expandable core, the first expandable chamber, the second expandable chamber, or a combination thereof is configured to engage an annulus fibrosus, the superior vertebra, the inferior vertebra, or a combination thereof when the expandable core, the first expandable chamber, and the second expandable chamber are inflated.

75. The method of claim 74, further comprising removing a first injection tube from the expandable core.

76. The method of claim 75, further comprising removing a second injection tube from the first expandable chamber.

77. The method of claim 76, further comprising removing a third injection tube from the second expandable chamber.

78. The method of claim 77, further comprising sealing the expandable core.

79. The method of claim 78, further comprising sealing the first expandable chamber.

80. The method of claim 79, further comprising sealing the second expandable chamber.

81. The method of claim 80, further comprising curing a material used to inflate the expandable core, the first expandable chamber, the second expandable chamber, or a combination thereof.

82. The method of claim 81, wherein the expandable core, the first expandable chamber, the second expandable chamber, or a combination thereof is inflated using an injectable extended use approved medical material.

83. The method of claim 82, wherein the injectable extended use approved medical material is a polymer material.

84. The method of claim 83, wherein the polymer material is a polyurethane material, a polyolefin material, a polyether material, a silicone material, or a combination thereof.

85. The method of claim 84, wherein the polyolefin material is a polypropylene, polyethylene, halogenated polyolefin, flouropolyolefin, or a combination thereof.

86. The method of claim 84, wherein the polyether material is polyetherketone (PEK), polyetheretherketone (PEEK), polyetherketoneketone (PEKK), polyaryletherketone (PAEK), or a combination thereof.

87. The method of claim 84, wherein the silicon material is silicone hydrogel.

88. The method of claim 82, wherein the injectable extended use approved medical material comprises sterile water, saline, sterile air, or a combination thereof.

89. A method of installing a nucleus implant within an intervertebral disc between an inferior vertebra and a superior vertebra of a patient, the method comprising:
implanting the nucleus implant within the intervertebral disc, wherein the nucleus implant includes an expandable core and an expandable chamber at least partially around the expandable core; and
inflating the expandable chamber;
inflating the expandable core within the expandable chamber, wherein a hardness of the expandable core when inflated is greater than or equal to a hardness of the expandable chamber when inflated.

90. The method of claim 89, wherein the expandable core, the expandable chamber, or a combination thereof is configured to engage an annulus fibrosus, the superior vertebra, the inferior vertebra, or a combination thereof when the expandable core and the expandable chamber are inflated.

91. The method of claim 90, further comprising removing a first injection tube from the expandable core.

92. The method of claim 91, further comprising removing a second injection tube from the expandable chamber.

93. The method of claim 92, further comprising sealing the expandable core.

94. The method of claim 93, further comprising sealing the expandable chamber.

95. The method of claim 94, further comprising curing a material used to inflate the expandable core, the expandable chamber, or a combination thereof.

96. The method of claim 95, wherein the expandable core, the expandable chamber, or a combination thereof is inflated using an injectable extended use approved medical material.

97. The method of claim 96, wherein the injectable extended use approved medical material is a polymer material.

98. The method of claim 97, wherein the polymer material is a polyurethane material, a polyolefin material, a polyether material, a silicone material, or a combination thereof.

99. The method of claim 98, wherein the polyolefin material is a polypropylene, polyethylene, halogenated polyolefin, flouropolyolefin, or a combination thereof.

100. The method of claim 98, wherein the polyether material is polyetherketone (PEK), polyetheretherketone (PEEK), polyetherketoneketone (PEKK), polyaryletherketone (PAEK), or a combination thereof.

101. The method of claim 98, wherein the silicon material is silicone hydrogel.

102. The method of claim 96, wherein the injectable extended use approved medical material comprises sterile water, saline, sterile air, or a combination thereof
